(19) United States
(12) Patent Application Publication
(10) Pub. No.: US 2018/0344253 A1
(43) Pub. Date: Dec. 6, 2018

(54) MEDICAL ROBOT

(71) Applicants: Fu Tai Hua Industry (Shenzhen) Co., Ltd., Shenzhen (CN); HON HAI PRECISION INDUSTRY CO., LTD., New Taipei (TW)

(72) Inventor: XUE-QIN ZHANG, Shenzhen (CN)

(21) Appl. No.: 15/788,586

(22) Filed: Oct. 19, 2017

(30) Foreign Application Priority Data
Jun. 2, 2017 (CN) 201710409047.7

(51) Int. Cl. A61B 5/00; A47B 83/02; A47B 83/04

(52) U.S. Cl. CPC A61B 5/6891; A47B 83/0215; A61B 5/021; Y10S 901/46; A47B 83/045

(57) ABSTRACT

A medical robot includes an instruction receiving unit for receiving input commands from a user, a number of measuring devices for measuring physiological parameters of the user, an output unit for outputting measurement results of the measuring devices measuring the physiological parameters, a storage device, and at least one processor. wherein the storage device stores one or more programs that when executed by the at least one processor, the one or more programs cause the at least one processor to control the measuring devices to measure the physiological parameters of the user according to the user's input commands on the instruction receiving unit, and to control the output unit to output voice messages to the user in response to the input commands.

### CROSS-REFERENCE TO RELATED APPLICATIONS

This application claims priority to Chinese Patent Application No. 201710409047.7 filed on Jun. 2, 2017, the contents of which are incorporated by reference herein.

### FIELD

The subject matter herein generally relates to medical devices, and more particularly to a medical robot.

### BACKGROUND

There are many all-in-one type medical devices on the market for personal use. However, these medical devices cannot interact with users.

### BRIEF DESCRIPTION OF THE DRAWINGS

Implementations of the present technology will now be described, by way of example only, with reference to the attached figures.

FIG. 1 is an assembled, isometric view of a medical robot, in accordance with an exemplary embodiment of the present application.

FIG. 2 is an assembled, isometric view of the medical robot in FIG. 1 in another state of use, in accordance with an exemplary embodiment of the present application.

FIG. 3 is a diagrammatic view of a medical robot, in accordance with an exemplary embodiment of the present application.

FIG. 4 is a diagrammatic view of function modules of a medical robot, in accordance with an exemplary embodiment of the present application.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. The drawings are not necessarily to scale and the proportions of certain parts may be exaggerated to better illustrate details and features. The description is not to be considered as limiting the scope of the embodiments described herein.

Several definitions that apply throughout this disclosure will now be presented.

The term “comprising” means “including, but not necessarily limited to”; it specifically indicates open-ended inclusion or membership in a so-described combination, group, series and the like.

In general, the word “module” as used hereinafter refers to logic embodied in hardware or firmware, or to a collection of software instructions, written in a programming language such as, for example, Java, C, or assembly. One or more software instructions in the modules may be embedded in firmware such as in an erasable-programmable read-only memory (EPROM). It will be appreciated that the modules may comprise connected logic units, such as gates and flip-flops, and may comprise programmable units, such as programmable gate arrays or processors. The modules described herein may be implemented as either software and/or hardware modules and may be stored in any type of computer-readable medium or other computer storage device.

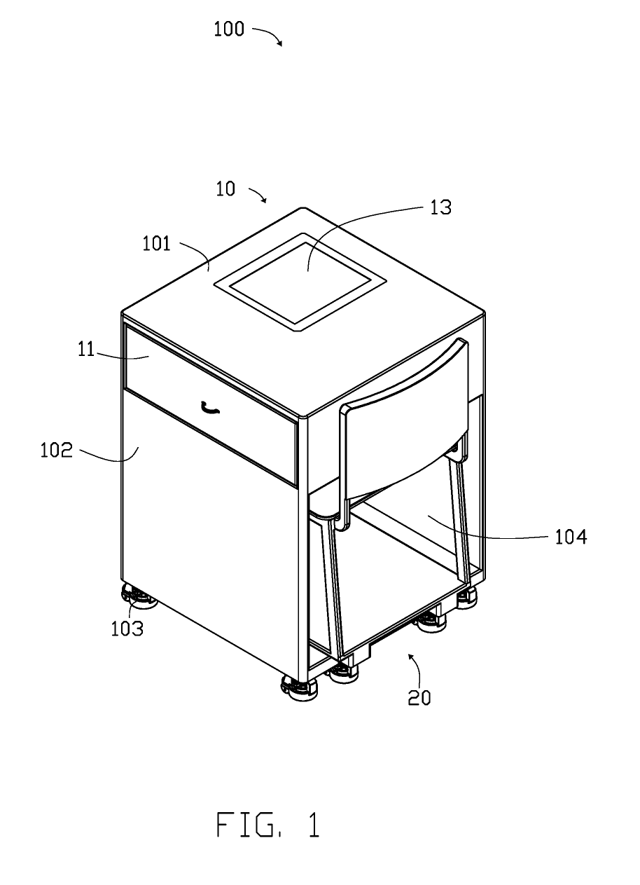

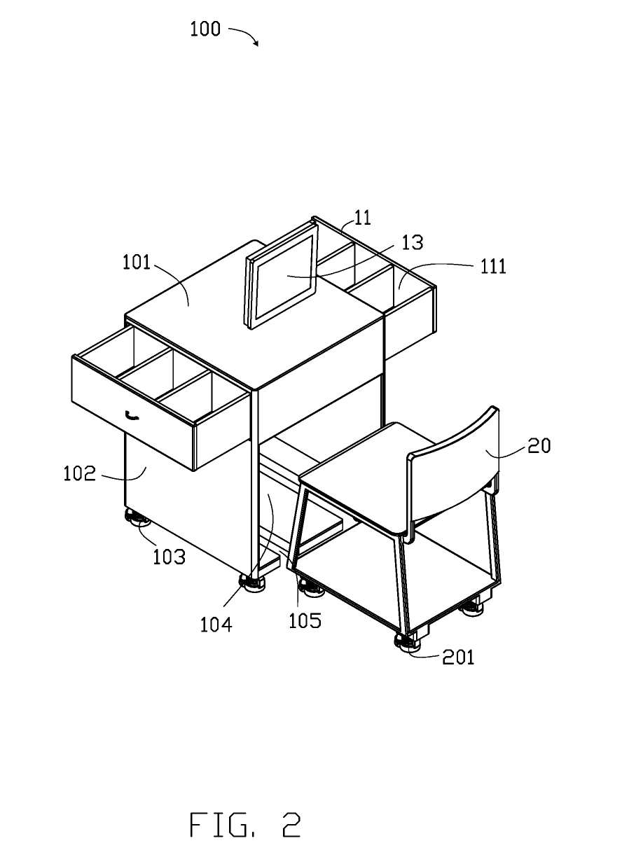

FIGS. 1 and 2 illustrate an exemplary embodiment of a medical robot 100. The medical robot 100 can include a desk 10 and a chair 20. The desk 10 can include a desktop 101 and a desk body 102. The desk 10 can include a plurality of wheels 103 attached to a bottom of the desk body 102. The desk 10 can be moved by the plurality of wheels 103. A receiving space 104 is defined in a bottom of the desk body 102 for receiving the chair 20 therein. The desk body 101 can include a plurality of sliding rails 105 for guiding the chair 20 to slide into or slide out of the receiving space 104. A bottom of the chair 20 can include a plurality of wheels 201 to allow the chair 20 to slide into and out of the receiving space 104. The desk 10 and the chair 20 can be made to have any shape according to the needs of a user. In other embodiments, the desk can have different colors, have different shapes, or be made from different materials such as plastic, wood, metal, rubber, or the like.

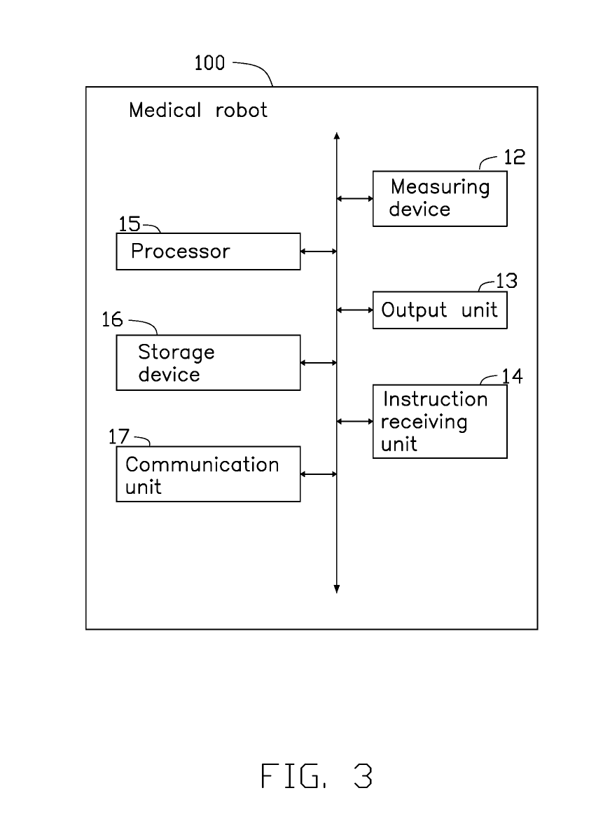

The desktop 101 can include a plurality of drawers 11. The plurality of drawers 11 can extend from and be received in the desktop 101 and receive a plurality of measuring devices 12 (shown in FIG. 3). The measuring devices 12 can measure different physiological parameters of a user, such as blood pressure, blood oxygen, blood sugar, body temperature, body mass, or the like. Each drawer 11 can be divided into a plurality of cubbies 111, and each cubby 111 can receive a corresponding measuring device 12 therein.

In at least one embodiment, besides being received in the drawers 11, some of the measuring devices 12 can be located in or on the chair 20. For example, the chair 20 can include a measuring device 12 for measuring body mass or body fat of a user when the user sits on the chair 20.

The desktop 101 can include an output unit 13 for outputting measurement results taken by the measuring devices 12. In at least one embodiment, the output unit 13 can display encyclopedic information, a facial expression, a video, or the like. The output unit 13 can be a display screen rotationally coupled to and embedded in the desktop 101, such that the output unit 13 can rotate out of the desktop 101 and rotate into the desktop 101. The output unit 13 can be made to be any shape according to user requirements, such as a square shape, a round shape, a heart shape, a face shape, a cartoon shape, or the like.

Referring to FIG. 3, the medical robot 100 can include an instruction receiving unit 14, a processor 15, a storage 16, and a communication unit 17. The processor 15 can be in communication with the plurality of measuring devices 12, the output unit 13, the instruction receiving unit 14, the processor 16, and the communication unit 17.

The instruction receiving unit 14 can receive input commands from a user. In at least one embodiment, the instruction receiving unit 14 can be a voice recognition device for receiving voice commands from a user. For example, the voice commands can include “move the table”, “slide out the chair”, “slide in the chair”, “open the drawers”, “close the drawers”, “measure blood pressure”, or the like. In other embodiments, the instruction receiving unit 14 can include a mouse, a keyboard, and other input apparatuses and/or methods. The instruction receiving unit 14 can further display a menu of items to be selected by a mouse and/or a keyboard.

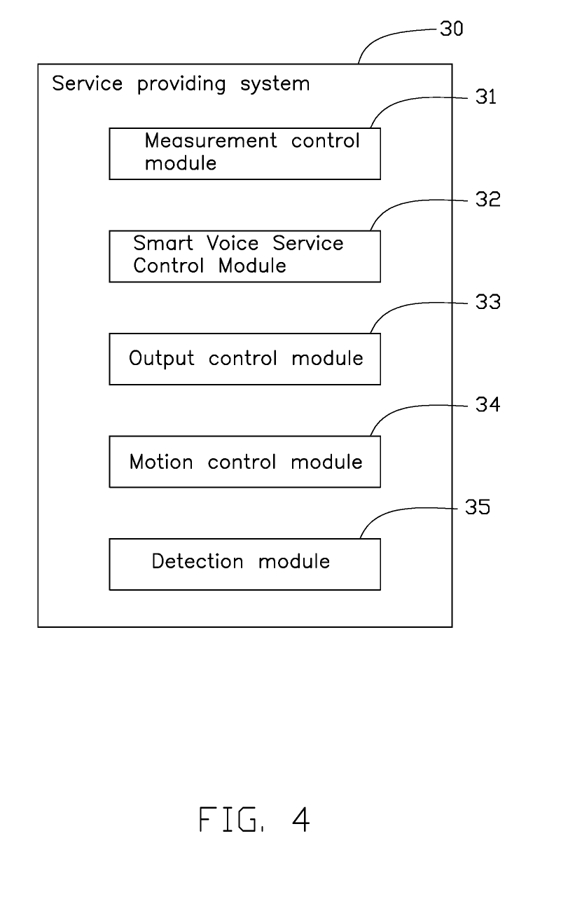

Referring to FIG. 4, the medical robot 100 can include a service providing system 30. The service providing system 30 can be composed of a plurality of modules including one or more software programs in the form of computerized codes stored in the storage 16. The computerized codes can include instructions executed by the processor 15 to provide functions for the modules. In at least one embodiment, the processor 15 can be a central processing unit, a single chip, or the like.

In at least one embodiment, the service providing system 30 can include a measurement control module 31, a smart voice service control module 32, an output control module 33, and a motion control module 34.

The measurement control module 31 can be used for controlling the measuring devices 12 to measure the physiological parameters of a user in response to input commands and for recording measurement results. For example, when a user selects an input command “measure body temperature” on the instruction receiving unit 14, the corresponding measuring device 12 can measure and record the body temperature of the user. In other embodiments, the measurement control module 31 can determine whether the user is near a certain measuring device 12 and then control the corresponding measuring device 12 to measure the corresponding physiological parameter(s) of the user. For example, when a forehead of a user is detected to be near the corresponding measuring device 12 for measuring body temperature, the measurement control module 31 can control the corresponding measuring device 12 for measuring body temperature to measure the body temperature of the user. In other embodiments, a user can manually control the measuring devices 12 to measure the physiological parameters.

In at least one embodiment, the measurement control module 31 is further used for comparing the measured physiological parameters of a user with pre-stored physiological parameters in the storage 16 to determine a comparison result. The pre-stored physiological parameters can be physiological parameters of a normal healthy person. In at least one embodiment, when the result of comparison shows that any of the measured physiological parameters of the user reaches a predetermined warning level stored in the storage device 16, the measurement control module 31 can automatically contact an emergency service for administering emergency aid to the user.

In at least one embodiment, the measurement control module 31 can communicate with a cloud server through the communication module 17 for uploading the measurement results for analysis.

The smart voice service control module 32 can respond to input commands from the user. In at least one embodiment, the smart voice service control module 32 can converse with the user, broadcast encyclopedic information, tell jokes and stories, broadcast music, or the like. In at least one embodiment, the smart voice service control module 32 can create a schedule of items according to input commands from the user and remind the user according to the schedule of items to take medicine, measure physiological parameters, or the like. In at least one embodiment, the smart voice service control module 32 can obtain the measurement results from the measuring devices 12 and provide health information, nutrition recommendations, medical advice, exercise instructions, or the like according to the measurement results. For example, if a measurement result shows that the user has high blood pressure, the smart voice service control module 32 can recommend nutritional information, exercise recommendations, and other health recommendations for reducing blood pressure.

In at least one embodiment, the smart voice service control module 32 can obtain voice messages from the cloud server through the communication unit 17.

The output control module 33 can control the output unit 13 to output the voice messages from the smart voice service control module 32. In at least one embodiment, the output unit 13 can include a loudspeaker.

The motion control module 34 can control the desk 10 and the chair 20 to move according to input commands from the user. For example, movements can include opening and closing the drawers 11, sliding out and sliding in the chair 20, moving the desk 10, opening and closing the output unit 13, or the like. Additionally, the desk 10 and the chair 20 can be moved manually.

The medical robot 100 can further include a detection module 35 for detecting whether a power level of the medical robot is below a threshold value. When it is detected that the power level is below the threshold value, the motion control module 34 can control the medical robot 100 to move to return to a predetermined location for charging.

The embodiments shown and described above are only examples. Even though numerous characteristics and advantages of the present technology have been set forth in the foregoing description, together with details of the structure and function of the present disclosure, the disclosure is illustrative only, and changes may be made in the detail, including in matters of shape, size and arrangement of the parts within the principles of the present disclosure up to, and including, the full extent established by the broad general meaning of the terms used in the claims.

What is claimed is:

1. A medical robot comprising:
an instruction receiving unit for receiving input commands from a user;
a plurality of measuring devices for measuring physiological parameters of the user;
an output unit for outputting measurement results of the measuring devices measuring the physiological parameters;
a storage device; and
at least one processor, wherein the storage device stores one or more programs that when executed by the at least one processor, the one or more programs cause the at least one processor to:
control the plurality of measuring devices to measure the physiological parameters of the user according to the input commands on the instruction receiving unit; and
control the output unit to output voice messages to the user in response to the input commands.

2. The medical robot of claim 1 further comprising a desk and a chair, the desk comprising a desktop and a desk body, a storage space being defined in the desk body for receiving the chair therein, the desk body comprising sliding rails for guiding the chair to slide into or out of the storage space; a plurality of wheels is attached to a bottom of the desk body and a bottom of the chair; the at least one processor is further configured to control the chair to slide into or slide out of the desk body in response to the input commands.

3. The medical robot of claim 2, wherein the desk comprises a plurality of drawers for receiving the plurality of measuring devices; the at least one processor controls the drawers to open or close according to the input commands.

4. The medical robot of claim 3, wherein the at least one processor is further configured to control the output device to provide voice messages to remind a user to follow a schedule of items set by the user.

5. The medical robot of claim 4, wherein the at least one processor is further configured to compare the measured physiological parameters of the user with preset physiological parameters stored in the storage device and control the output unit to output a comparison result.

6. The medical robot of claim 5, wherein the at least one processor is further configured to determine whether any of the measured physiological parameters of a user reach a predetermined warning level stored in the storage device and automatically contact an emergency service when it is determined that any of the measured physiological parameters reach the warning level.

7. The medical robot of claim 6, wherein the at least one processor is further configured to determine whether a power level of the medical robot is lower than a predetermined threshold and control the medical robot to return to a predetermined location to be charged.

8. The medical robot of claim 1 further comprising a communication unit for communicating with a cloud server; the communication unit can obtain voice messages from the cloud server and upload the measured physiological parameters to the cloud server for analysis.

9. The medical robot of claim 2, wherein the output unit is a display panel rotationally embedded in the desktop of the desk.
